(19) United States
(12) Patent Application Publication
(10) Pub. No.: US 2026/0259223 A1
(43) Pub. Date: Sep. 3, 2026

(54) Collagen Type VI Alpha-6 Assay

(71) Applicant: Nordic Bioscience A/S, Herlev (DK)

(72) Inventors: Signe Holm Nielsen, Frederiksberg (DK); Cecilie Hausgaard Møller, København SV (DK); Anne-Christine Bay-Jensen, København S (DK); Morten Asser Karsdal, København Ø (DK)

(73) Assignee: Nordic Bioscience A/S, Herlev (DK)

(21) Appl. No.: 18/994,439

(22) PCT Filed: Jul. 17, 2023
(86) PCT No.: PCT/EP2023/069840

§ 371 Date: Jan. 14, 2025

(30) Foreign Application Priority Data
Jul. 18, 2022 (GB) 2210527.4

(51) Int. Cl. G01N 33/68; C07K 16/18; G01N 33/5751

(52) U.S. Cl. CPC G01N 33/6881; C07K 16/18; G01N 33/5751; G01N 2333/78; G01N 2470/04; G01N 2470/10; G01N 2800/20; G01N 2800/202; G01N 2800/205

(57) ABSTRACT

Disclosed herein are methods of immunoassay for detecting and/or monitoring a skin disease in a patient and/or determining the severity of a skin disease in a patient. The methods detect and quantify the levels of a biomarker that has an N-terminus sequence of collagen type VI alpha-6, in a blood-based sample, such as whole blood, plasma or serum, from the patient. Also disclosed are antibodies suitable for use in the methods and immunoassay kits suitable for carrying out the methods.

### FIELD OF INVENTION

The present invention relates to a method of immunoassay for detecting and/or monitoring a skin disease in a patient and/or determining the severity of a skin disease in a patient. The method comprises detecting and quantifying the levels of a biomarker, comprising an N-terminus sequence of collagen type VI alpha-6, in a blood-based sample, such as whole blood, plasma or serum, from the patient. The present invention also relates to antibodies suitable for use in said methods and immunoassay kits suitable for carrying out said methods. The skin disease may, in particular, be a skin disease selected from atopic dermatitis, melanoma, psoriasis, hidradenitis suppurativa, systemic sclerosis or systemic lupus erythematosus.

### BACKGROUND

Extracellular matrix (ECM) remodelling of the skin is a continuous process necessary to maintain tissue homeostasis. The skin can be divided into three layers; epidermis, dermis and subcutis, which all have distinct tissue architecture and function (1,2). The dermis provides tensile strength, elasticity and resilience to the skin, in addition to being the layer where fibroblasts play a key role in ECM synthesis and maintenance of tissue structure (3,4). The ECM of the dermis can be divided into the papillary and reticular dermal ECM, and is composed by matricellular proteins (COMP, SPARC, Thrombospondin-1, periostin, tenascin C and X), proteoglycans (Decorin, versican, biglycan, fibromodulin and lumican), collagens (I, III, V, VI, XII, XIV and XV), fibrillin microfibrils (Fibrillin-1 and -2) and elastic fiber proteins (elastin, EMLIN-1 and 2, LTBP-4, fibulin-4 and -5) (2). Dysregulation of the papillary and reticular dermal ECM remodelling is a key event in the pathology of dermatological conditions, including atopic dermatitis and psoriasis (1,2,5).

Generally, the importance of type VI collagen in maintenance of tissue homeostasis in the skin has been described in the literature. Type VI collagen is characterized as a beaded filament collagen, found in the papillary and reticular dermal ECM, were it forms a microfibrillar network (6). Six different chains of type VI collagen (α1, α2, α3, α4, α5, α6) have been identified and are expressed across connective tissues (7). In vitro experiments have demonstrated how type VI collagen α1-chain is important for ECM assembly in human dermal fibroblasts, and showed loss of type VI collagen resulted in loss of fibroblast motility (8). In skin pathologies, the α3-chain, measured by the serum-biomarker PRO-C6, has previously been associated with progression of systemic sclerosis (9). The role of the type VI collagen α6-chain gene (COL6α6) in patients with atopic dermatitis (AD) has been investigated using whole-exome sequencing, transcriptomics, immunohistochemical staining and mRNA analysis (10-12). Using immunohistochemical staining of skin samples it was shown in one study that the expression of COL6α6 was decreased in epidermis and increased in the dermis of AD patients, decreased in epidermis and dermis of psoriasis patients, increased in the epidermis and decreased in the dermis of a patient with papular urticaria, and decreased in the epidermis of patients with pityriasis rosea, all as compared to healthy controls (10). In addition, it was shown via mRNA analysis that the total mRNA expression of COL6α6 was increased in skin samples (comprising both the dermis and epidermis) from AD patients as compared to health controls, and that COL6α6 mRNA expression is suppressed in human keratinocytes exposed to the inflammatory cytokines IL-4 and IL-13 (10).

### SUMMARY

The applicant has now developed and validated a competitive ELISA for detecting and quantifying the levels of a biomarker, comprising the N-terminus amino acid sequence DSGPEYADVV of the collagen type VI α6-chain (COL6α6), in blood-based samples from patients, and has demonstrated the use of this immunoassay for detecting, monitoring and/or determining the severity of various skin diseases. The immunoassay methods, kits and monoclonal antibodies disclosed herein can therefore be used for analysing blood-based samples from a patient in order to conveniently detect, monitor and/or assess such diseases.

Accordingly, in a first aspect the present invention provides a method of immunoassay comprising:
i) contacting a patient sample, selected from blood, serum or plasma, with a monoclonal antibody that specifically binds to the N-terminus amino acid sequence DSGPEYADVV (SEQ ID NO: 1) (also referred to herein as “C6A6” and/or “the target sequence”); and
ii) detecting and determining the amount of binding between said monoclonal antibody and peptides in the sample.

In preferred embodiments, the method is a method of immunoassay for detecting and/or monitoring a skin disease and/or determining the severity of a skin disease in a patient, the method further comprising:
iii) correlating said amount of binding with values associated with normal healthy subjects and/or values associated with known disease severity and/or values obtained from said patient at a previous time point and/or with a predetermined cut-off value.

As used herein the term “N-terminus” refers to a N-terminal peptide sequence at the extremity of a polypeptide, i.e. at the N-terminal end of the polypeptide, and is not to be construed as meaning in the general direction thereof.

As used herein, the terms “peptide” and “polypeptide” are used synonymously.

In preferred embodiments, the monoclonal antibody does not specifically bind to a peptide having the N-terminus amino acid sequence QDSGPEYADVV (SEQ ID NO: 2) (i.e. an N-extended version of the target sequence).

In preferred embodiments, the monoclonal antibody does not specifically bind to a peptide having the N-terminus amino acid sequence SGPEYADVV (SEQ ID NO: 3) (i.e. an N-truncated version of the target sequence).

In preferred embodiments, the monoclonal antibody is raised against a synthetic peptide having the N-terminus amino acid sequence DSGPEYADVV (SEQ ID NO: 1). For example, the monoclonal antibody may be raised by: (a) immunizing a rodent (or other suitable mammal) with a synthetic peptide comprising the N-terminus sequence DSGPEYADVV (SEQ ID NO: 1), which peptide may optionally be linked at its C-terminus to an immunogenic carrier protein (such as keyhole limpet hemocyanin); (b) isolating and cloning a single antibody producing cell; and (c) assaying the resulting monoclonal antibodies to ensure that they have the desired specificity.

As used herein the term “monoclonal antibody” refers to both whole antibodies and to fragments thereof that retain the binding specificity of the whole antibody, such as for example a Fab fragment, F(ab′) 2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4 in the case of humans and IgGI, IgG2a, IgG2b, IgG2c and IgG3 in the case of mice. The monoclonal antibody may preferably be of the IgG isotype, including any one of the IgG subclasses (e.g, IgGI, IgG2, IgG3 or IgG4 in the case of human antibodies).

The CDR of an antibody can be determined using methods known in the art such as that described by Kabat et al. Antibodies can be generated from B cell clones as described in the examples. The isotype of the antibody can be determined by ELISA specific for IgM, IgG or IgA isotype (human or mouse), or subclass (human or mouse). The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In certain exemplary embodiments, the monoclonal antibody may preferably comprise one or more complementarity-determining regions (CDRs) selected from:

CDR-L1:
(SEQ ID NO: 4)
KASQNVGTDVV
CDR-L2:
(SEQ ID NO: 5)
SASYRYS
CDR-L3:
(SEQ ID NO: 6)
QHYDNYPLT
CDR-H1:
(SEQ ID NO: 7)
SYAMS
CDR-H2:
(SEQ ID NO: 8)
SVTSGGTHYLDSVKG
CDR-H3:
(SEQ ID NO: 9)
GVSFAY

Preferably the monoclonal antibody comprises at least 2, 3, 4, 5 or 6 of the above listed CDR sequences.

Preferably the monoclonal antibody has a light chain variable region comprising the CDR sequences:

CDR-L1:
(SEQ ID NO: 4)
KASQNVGTDVV
CDR-L2:
(SEQ ID NO: 5)
SASYRYS
and
CDR-L3:
(SEQ ID NO: 6)
QHYDNYPLT.

Preferably the monoclonal antibody has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO: 10)
KASQNVGTDVVWYQQRPGQSPKALMYSASYRYSGVPDRFTGSGSGTD
FTLTISNVQSEDLAEYFCQHYDNYPLT

Preferably the monoclonal antibody has a heavy chain variable region comprising the CDR sequences:

CDR-H1:
(SEQ ID NO: 7)
SYAMS
CDR-H2:
(SEQ ID NO: 8)
SVTSGGTHYLDSVKG
and
CDR-H3:
(SEQ ID NO: 9)
GVSFAY.

Preferably the monoclonal antibody has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO: 11)
SYAMSWVRQTPEKRLEWVASVTSGGTHYLDSVKGRFTISRDIARNIL
YLQMSSLRSEDTAMYFCARGVSFAY

Preferably, the monoclonal antibody comprises the light chain variable region sequence:

(SEQ ID NO: 12)
DIVMTQSQKFMSTSVGDRVSVTCKASQNVGTDVVWYQQRPGQSPKAL
MYSASYRYSGVPDRFTGSGSGTDFTLTISNVQSEDLAEYFCQHYDNY
PLTFGAGTKLELK

(CDRs bold and underlined; Framework sequences in italics)
and/or the heavy chain variable region sequence:

(SEQ ID NO: 13)
EVKLVESGGDLVKPGGSLKLSCAASGFTFTSYAMSWVRQTPEKRLEW
VASVTSGGTHYLDSVKGRFTISRDIARNILYLQMSSLRSEDTAMYFC
ARGVSFAYWGQGTLVTVSA

(CDRs bold and underlined; Framework sequences in italics)

As used herein, the framework amino acid sequences between the CDRs of an antibody are “substantially identical” or “substantially similar” to the framework amino acid sequences between the CDRs of another antibody if they have at least 70%, 80%, 90% or at least 95% similarity or identity. The similar or identical amino acids may be contiguous or non-contiguous. The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In preferred embodiments the patient sample is serum or plasma.

In preferred embodiments the skin disease is atopic dermatitis, melanoma, psoriasis, hidradenitis suppurativa or systemic lupus erythematosus.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to thereby determine the quantity of peptide to which the antibody specifically binds in the patient sample. In the Examples set out below, an ELISA method is used in which spectrophotometric analysis is used to measure the amount of binding both in the patient samples and when producing the calibration curve. However, any suitable analytical method can be used.

As used herein the term “predetermined cut-off value” means an amount of binding that is determined statistically to be indicative of a high likelihood of a disease or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease or said particular severity thereof.

As used herein, the term “values associated with normal healthy subjects” means standardised quantities of binding determined by the method described supra for samples from subjects considered to be healthy, i.e. without disease; and the term “values associated with known disease severity” means standardised quantities of binding determined by the method described supra for samples from patients known to have disease of a known severity.

In a second aspect, the present invention provides a method of treating a skin disease in a patient in need thereof, the method comprising:
(a) carrying out a method of immunoassay in accordance with the first aspect of the present invention on a blood, serum or plasma sample from a patient in order to detect whether the patient has a skin disease and/or determine the severity of a skin disease in the patient; and
(b) administering to the patient a medicament for the treatment of said skin disease if it is determined in step (a) that the patient has said skin disease or a particular severity thereof.

The medicament may be any medicament suitable for treating the skin disease in question. The medicament may for example comprise or consist of one or more topical medicaments, one or more systemic medicaments, or combinations thereof. Topical medicaments may for example be formulated as creams, foams, gels, lotions, or ointments for administration to the area or areas of skin requiring treatment. Systemic medicaments may for example be formulated for enteral or parenteral administration.

For example, where the skin disease is atopic dermatitis, suitable topical medicaments may be selected from moisturisers, topical corticosteroids (e.g. hydrocortisone), topical immunosuppressants such as for example topical calcineurin inhibitors (e.g. tacrolimus and pimecrolimus), and PDE-4 inhibitors (e.g. crisaborole); and suitable systemic medicaments may be selected from systemic immunosuppressants (e.g. ciclosporin, methotrexate, interferon gamma-1b, mycophenolate mofetil, and azathioprine), biologics (e.g. monoclonal antibodies such as dupilumab and tralokinumab), and JAK inhibitors (e.g. abrocitinib and upadacitinib).

Where the skin disease is melanoma, suitable medicaments may for example be selected from chemotherapeutic agents (e.g. dacarbazine, temozolomide, cisplatin, carboplatin and paclitaxel), BRAF inhibitors (e.g. vemurafenib and dabrafenib), MEK inhibitors (e.g. trametinib), C-Kit inhibitors, NRAS inhibitors, cytokines (e.g. IL-2 and IFN-α), immune check point inhibitors (e.g. anti-CTLA-4 monoclonal antibodies, TLR agonists, CD40 agonists, anti-PD-1 antibodies and PD-L1 antibodies), and adoptive cell transfer.

Where the skin disease is psoriasis, suitable topical medicaments may for example be selected from moisturisers, topical corticosteroids (e.g. hydrocortisone), vitamin D analogues (e.g. paricalcitol), and topical immunosuppressants such as for example topical calcineurin inhibitors (e.g. tacrolimus and pimecrolimus); and suitable systemic medicaments may be selected from systemic immunosuppressants (e.g. ciclosporin and methotrexate), fumarates (e.g. dimethyl fumarate), retinoids, and biologics (.g. monoclonal antibodies such as ixekizumab, secukinumab, brodalumab, guselkumab, certolizumab, and ustekinumab).

Where the skin disease is hidradenitis suppurativa, suitable medicaments may for example be selected from topical antibiotics (e.g. topical clindamycin), topical retinoids (e.g. isotretinoin), oral antibiotics (e.g. rifampicin, clindamycin, tetracycline and minocycline), corticosteroids administered via intralesional injection, antiandrogenic medications (e.g. spironolactone, flutamide, cyproterone acetate, ethinylestradiol, finasteride, dutasteride, and metformin), and TNF inhibitors (e.g. etanercept) and anti-TNF-alpha monoclonal antibodies (e.g. infliximab and adalimumab) administered intravenously or via subcutaneous injection or infusion.

Where the skin disease is systemic lupus erythematosus, suitable medicaments may for example be selected from nonsteroidal anti-inflammatory drugs (NSAIDs), corticosteroids, antimalarial drugs (hydroxychloroquine), BLyS-specific inhibitors (e.g. belimumab), and immunosuppressive agents (e.g. prednisone, mycophenolic acid, tacrolimus, methotrexate and azathioprine).

In certain embodiments, step (a) of the method may comprise carrying out the method of immunoassay in accordance with the first aspect of the present invention on the blood, serum or plasma sample from a patient in order to determine the severity of a skin disease in the patient, and step (b) of the method may comprise administering to the patient a medicament for the treatment of said skin disease only if it is determined in step (a) that the patient has a particular severity of said skin disease. For example, the method may involve administering a topical medicament if the severity of the disease is at or below a particular level, and administering a systemic medicament if the severity of the disease is above said level. In one particular embodiment, the skin disease is atopic dermatitis, and step (b) comprises administering a topical medicament to the patient if it is determined in step (a) that the patient has mild or moderate atopic dermatitis, and administering a systemic medicament to the patient if it is determined in step (a) that the patient has severe atopic dermatitis.

In a third aspect, the present invention provides an immunoassay kit comprising a monoclonal antibody that specifically binds to the N-terminus amino acid sequence DSGPEYADVV (SEQ ID NO: 1), and at least one of;
a streptavidin coated well plate;
a biotinylated peptide DSGPEYADVV-L-Biotin (SEQ ID NO: 14), wherein L is an optional linker;
a secondary antibody for use in a sandwich immunoassay;
a calibrator protein comprising the N-terminus amino acid sequence DSGPEYADVV (SEQ ID NO: 1);
an antibody biotinylation kit;
an antibody HRP labelling kit;
an antibody radiolabelling kit; and
an assay visualisation kit.

In a preferred embodiment, the monoclonal antibody does not specifically bind to a peptide having the N-terminus amino acid sequence QDSGPEYADVV (SEQ ID NO: 2).

In a preferred embodiment, the monoclonal antibody does not specifically bind to a peptide having the N-terminus amino acid sequence SGPEYADVV (SEQ ID NO: 3).

In a preferred embodiment, the monoclonal antibody is raised against a synthetic peptide having the N-terminus amino acid sequence DSGPEYADVV (SEQ ID NO: 1).

The immunoassay kit according to the third aspect of the invention is, in particular, suitable for use in carrying out the method according to first aspect of the invention. Further preferred embodiments and features of the immunoassay kit according to the third aspect will therefore be apparent from the above discussion of the preferred embodiments of the method according to the first aspect.

In a fourth aspect, the present invention provides a monoclonal antibody that specifically binds to the N-terminus amino acid sequence DSGPEYADVV (SEQ ID NO 1).

In a preferred embodiment, the monoclonal antibody does not specifically bind to a peptide having the N-terminus amino acid sequence QDSGPEYADVV (SEQ ID NO: 2).

In a preferred embodiment, the monoclonal antibody does not specifically bind to a peptide having the N-terminus amino acid sequence SGPEYADVV (SEQ ID NO: 3).

In a preferred embodiment, the monoclonal antibody is raised against a synthetic peptide having the N-terminus amino acid sequence DSGPEYADVV (SEQ ID NO: 1).

The monoclonal antibody according to the fourth aspect of the invention is, in particular, suitable for use in the method according to first aspect of the invention. Further preferred embodiments and features of the monoclonal antibody according to the fourth aspect will therefore be apparent from the above discussion of the preferred monoclonal antibodies for use in the method according to the first aspect.

### FIGURES

FIG. 1: A) An overview of the primary structure of type VI collagen alpha-chains, showing the domains of all six alpha-chains. As shown, the antibody used in the C6A6 assay targets the C6A6 target sequence at N-terminus of the type VI collagen alpha-6 chain (COL6α6). B) A sequence alignment of the N-terminus sequences of COL6α6 in human, mouse, bovine and rat species, with the C6A6 target sequence being highlighted (black box). The sequences were aligned using Uniprot. C) Specificity of the C6A6 assay. Reactivity towards the standard peptide (DSGPEYADVV (SEQ ID NO: 1)), truncated peptide (SGPEYADVV (SEQ ID NO: 3)), elongated peptide (QDSGPEYADVV (SEQ ID NO: 2)) and non-sense standard peptide (YRDDLKKLLE (SEQ ID NO: 17)) is shown. No background signal was detected when coating with a non-sense coating peptide (YRDDLKKLLE-Biotin (SEQ ID NO: 16)). Signals are shown as relative luminescence (RLU) per second, as a function of standard peptide.

FIG. 2: Results from cohort 1. Serum levels of C6A6 were assessed in healthy donors (n=20), and in patients with atopic dermatitis (n=20), melanoma (n=20), psoriasis (n=20), hidradenitis suppurativa (n=6), systemic sclerosis (n=14), systemic lupus erythematosus (n=12), urticaria (n=19) and vitiligo (n=20). Mean with 95% confidence interval (CI). Data was analyzed using an ANCOVA corrected for age and gender. Data are depicted as mean±95% Cl.

FIG. 3: Results from cohort 2. Serum levels of C6A6 in healthy donors (n=22) and patients with atopic dermatitis (n=158). Mean with 95% confidence interval. Data was analyzed using an ANCOVA corrected for age. Data are depicted as mean±95% Cl.

FIG. 4: Results from cohort 2. Serum levels of C6A6 stratified into disease severity and treatment. A) Levels of C6A6 in patients with mild and moderate AD (SCORAD; 0-50), versus patients with severe AD (SCORAD; <50). B) Levels of C6A6 in AD patients receiving no treatment versus AD patients receiving immunosuppressant treatment. C) Levels of C6A6 in patients with mild and moderate AD patients receiving no treatment versus in patients with mild and moderate AD patients receiving immunosuppressant treatment. D) Levels of C6A6 in patients with severe AD receiving no treatment versus patients with severe AD receiving immunosuppressant treatment. Mean with 95% confidence interval. Data was analyzed using an ANCOVA corrected for age. Data are depicted as mean±95% Cl.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

All reagents used were high quality chemicals from Merck (Whitehouse Station, NJ, USA) and Sigma (St. Louis MO, USA) unless stated otherwise. All synthetic peptides used for antibody production and assay validation were purchased from Genscript (Piscataway, NJ, US) (Table 1).

TABLE 1
Sequences of the synthetic peptides used for
monoclonal antibody production, assay development
and validation.

| Peptide type | Sequence |
|---|---|
| Immunogenic peptide | DSGPEYADVV-GGC-KLH* |
| | (SEQ ID NO: 15) |
| Standard peptide | DSGPEYADVV |
| (Selection peptide) | (SEQ ID NO: 1) |
| Elongated standard | QDSGPEYADVV |
| peptide (Elongated | SEQ ID NO: 2) |
| selection peptide) | |
| Truncated standard | SGPEYADVV |
| peptide (Truncated | (SEQ ID NO: 3) |
| selection peptide) | |
| Non-sense coating | YRDDLKKLLE-Biotin |
| and standard peptide | (SEQ ID NO: 16) |
| | and YRDDLKKLLE |
| | (SEQ ID NO: 17) |

*Keyhole Limpet Hemocyanin.

### Monoclonal Antibody Development, Production, and Characterization

The amino acid sequence 20′ ⬇DSGPEYADVV '30 (SEQ ID NO: 1) (also referred to herein as “C6A6” and/or “the target sequence”) in the human α6 chain of type VI collagen (COL6α6), which sequence forms the N-terminus of the collagen type VI α6 chain after cleavage and removal of the signal peptide, was used for generation of monoclonal antibodies (mAbs). Immunization was initiated by subcutaneous injection of 200 μl emulsified antigen and 100 μg immunogenic peptide (DSGPEYADVV-GGC-KLH (SEQ ID NO: 15)) in 4- to 6-week-old Balb/C mice using Stimmune (Thermo Fisher). The immunizations were repeated every second week until stable serum antibody titer levels were reached. The mouse with the highest serum titer was selected for fusion and rested for a month. Subsequently, the mouse was boosted intravenously with 50 μg immunogenic peptide in 100 μl 0.9% NaCl solution 3 days before isolation of the spleen for cell fusion. To produce hybridoma cells, the mouse spleen cells were fused with SP2/0 myeloma cells as described by Gefter et al. (13). Subsequently, the clones were plated into 96-well microtiter plates for further growth, and the limiting dilution method was applied to promote monoclonal growth. An indirect ELISA performed on streptavidin-coated plates was used for the screening of supernatant reactivity. DSGPEYADVV-K-Biotin (SEQ ID NO: 18) was used as screening peptide, while the standard peptide DSGPEYADVV (SEQ ID NO: 1) was used to further test the specificity of the clones. Supernatant was collected from the hybridoma cells and purified using HiTrap affinity columns (GEHealthcare Life Science, Little Chalfront, Buckinghamshire, UK) according to manufacturer's instructions and antibody isotype was determined using Rapid ELISA Mouse monoclonal antibody Isotyping Kit (Invitrogen, Carlsbad, CA, USA) following the manufacturer's protocol.

Native reactivity was assessed using human serum, citrate plasma, heparin plasma, EDTA plasma and rat serum, purchased from a commercial supplier (Valley Biomedical, Winchester, VA). The mAb was selected to specifically recognize the standard peptide (DSGPEYADVV (SEQ ID NO: 1)), and not an elongated or truncated sequence of one amino acid (QDSGPEYADVV (SEQ ID NO: 2) and SGPEYADVV (SEQ ID NO: 3), respectively).

The isotype, sequence and CDRs of the monoclonal antibody chosen for production and assay development were determined. The sequence of the chains are as follows (CDRs underlined and in bold; N-terminus signal peptide and C-terminus Constant region in italics):

Heavy Chain Sequence (Mouse IgG1 isotype)
(SEQ ID NO: 19)
MNFGFSLIFLVLVLNGVQCEVKLVESGGDLVKPGGSLKLSCAASGF
TFTSYAMSWVRQTPEKRLEWVASVTSGGTHYLDSVKGRFTISRDIA
RNILYLQMSSLRSEDTAMYFCARGVSFAYWGQGTLVTVSAAKTTPP
SVYPLAPGSAAQTNSMVTLGCLVKGYFPEPVTVTWNSGSLSSGVHT
FPAVLQSDLYTLSSSVTVPSSTWPSETVTCNVAHPASSTKVDKKIV
PRDCGCKPCICTVPEVSSVFIFPPKPKDVLTITLTPKVTCVVVDIS
KDDPEVQFSWFVDDVEVHTAQTQPREEQFNSTFRSVSELPIMHQDW
LNGKEFKCRVNSAAFPAPIEKTISKTKGRPKAPQVYTIPPPKEQMA
KDKVSLTCMITDFFPEDITVEWQWNGQPAENYKNTQPIMDTDGSYF
VYSKLNVQKSNWEAGNTFTCSVLHEGLHNHHTEKSLSHSPGK
Light Chain Sequence (Mouse Kappa Isotype)
(SEQ ID NO: 20)
MESQTQVFVYMLLWLSGVDGDIVMTQSQKFMSTSVGDRVSVTCKAS
QNVGTDVVWYQQRPGQSPKALMYSASYRYSGVPDRFTGSGSGTDFT
LTISNVQSEDLAEYFCQHYDNYPLTFGAGTKLELKRADAAPTVSIF
PPSSEQLTSGGASVVCFLNNFYPKDINVKWKIDGSERQNGVLNSWT
DQDSKDSTYSMSSTLTLTKDEYERHNSYTCEATHKTSTSPIVKSFN
RNEC

### C6A6 Assay Development

The development of a competitive chemiluminescence immunoassay (CLIA) targeting the C6A6 target sequence included several preliminary optimizing experiments where reagents, concentrations, incubation-time and -temperature were analyzed by several tests. The final C6A6 competitive ELISA procedure was as follows: A 96-well streptavidin-coated white microplate (Greiner Bio-One, Kremsmünster, Austria) was coated with 3 ng/mL biotinylated synthetic peptide (DSGPEYADVV-K-Biotin (SEQ ID NO: 18)) dissolved in assay buffer (10 mM phosphate buffered saline (PBS), 1% bovine serum albumin, 0.1% Tween-20, 0.36% Bronidox, 4 g/L NaCl, adjusted to pH 7.4 at 20° C.) and incubated for 30 min at 20° C. with constant shaking (300 rpm) in darkness. Next, 20 μL/well of standard peptide (100 ng/ml) and samples were added to the appropriate wells, followed by the addition of 100 μL/well of HRP-labelled antibody (generated as described above) diluted in assay buffer to the concertation of 200 ng/ml and incubated for 1 h at 20° C. with constant shaking (300 rpm) in darkness. After each incubation step, wells were washed five times with standard washing buffer (20 mM Tris, 50 mM NaCl, pH 7.2). The chemiluminescence substrate (Roche, BM Chemiluminescence ELISA substrate (POD), Basel, Switzerland) working solutions were mixed 15 min before use and 100 μL/well were added to plate and incubated for 3 min at 20° C. with constant shaking (300 rpm) in darkness. The relative light units were measured at all wavelengths within 5 min on a microplate luminometer reader (SpectraMax M5, Molecular Devices, CA, USA). A standard curve was plotted using a 4-parameter logistic curve fit Y=(A−D)/(1+ (x/C)\hat{ }B)+D, where R>0.9. Data were analyzed using the SoftMax Pro version 7.0.3 software.

### Technical Evaluation

Two-fold dilutions of four human serum and two rat serum samples were used to assess linearity. Linearity was calculated as a percentage of recovery of the undiluted sample. Antibody specificity was calculated as percentage of signal inhibition by two-fold diluted standard peptide (DSGPEYADVV (SEQ ID NO: 1)), elongated peptide (QDSGPEYADVV (SEQ ID NO: 2)), truncated peptide (SGPEYADVV (SEQ ID NO: 3)), and non-sense peptide (YRDDLKKLLE (SEQ ID NO: 17)). The intra- and inter-assay variation was determined by 10 independent runs of five quality controls and two kit controls run in double determinations. Accuracy of the assay was measured in healthy human serum samples spiked with standard peptide and a serum sample with a known high COL6α6 concentration and calculated as the percentage recovery of the measured value and the expected concentration of the peptide or the serum sample with high COL6α6 plus the concentration of the analyte in serum. Analytical interference was performed by adding a low/high content of hemoglobin (2.50/5 mg/mL), lipemia/lipids (1.50/5 mg/mL) and biotin (3/9 ng/mL) to a serum sample of known concentration. Recovery percentage was calculated with the normal serum sample as reference. The normal reference levels for hemoglobin, lipidemia/lipids and biotin were 0-10 mg/dl (0-0.00161 mmol/L), <150 mg/dl (<1.6935 mmol/L) and 0.221-3.004 ng/ml, respectively. The interference was calculated as the percentage recovery of the analyte in non-spiked serum. The measurement range was defined as the range between lower limit of measurement range (LLMR) and the upper limit of measurement range (ULMR), which were determined from 10 independent runs with the standard peptide. Measurements below LLMR or above ULMR were assigned the value of LLMR/ULMR respectively. IC50 (half-maximal inhibition concentration) was determined from the standard curve. The analyte stability was examined through temperature tests and repeated freeze-thaw cycles of serum samples. The temperature tests included different time point and temperatures where C6A6 target sequence levels were measured in three human serum samples after 0, 2, 4, 24, and 48 hours incubation at either 4° C. or 20° C. The recovery was estimated with 0 hours sample as a reference. Furthermore, the effect of four repeated freeze/thaw cycles of three serum samples was assessed where freeze/thaw recovery was calculated with the zero cycle samples as a reference. Each sample were run in double determination.

### Biological Evaluation of the C6A6 Assay and Patient Demographics

The biological utility of the C6A6 assay (described above) was evaluated in serum samples from two cross-sectional studies. The first cohort (Cohort 1) was obtained from the commercial vendor Proteogenex (Culver City, CA, USA), while the second cohort (Cohort 2) was obtained from Department of Dermatology, Bispebjerg Hospital, University of Copenhagen, Denmark. The healthy donor samples were acquired from BiolVT and Lee Biosolutions.

Cohort 1 included patients with atopic dermatitis (n=20), melanoma (n=20), psoriasis (n=20), hidradenitis suppurativa (n=6), systemic sclerosis (n=18), systemic lupus erythematosus (n=12), urticaria (n=19) and vitiligo (n=20) and matched healthy donors (n=24) with no symptomatic or chronic disease. Cohort 2 included 158 patients with atopic dermatitis and 22 healthy age, gender, and ethnicity matched controls. Serum samples were collected from January 2012 and June 2018 (8). The patients met the criteria defined by Hannifin and Rajka 1980. The AD diagnosis according to criteria was ensured by a senior physician. Severity was assessed by SCORAD, which is a commonly used severity index for AD (range 0-103 points; high score indicates severe disease). Patients were divided into disease severity groups based on SCORAD into mild (SCORAD<25), moderate (SCORAD range 25-50), and severe disease (SCORAD<50) (2,3). Out of 158 patients, 47 patients received topical immunosuppressant treatment. The treatments were topical calcineurin inhibitors. Only age and gender were available for the healthy donor subjects.

Samples from both cohorts were collected after informed consent and approval by the local Ethical Committee and in compliance with the Helsinki Declaration of 1975. Serum samples were obtained and stored at −80° C.

### Ethical Statement

All animals were treated according to the guidelines for animal welfare. Monoclonal antibody production in mice was approved by the Danish National Authority (The Animal Experiments Inspectorate) under approval number 2013-15-2934-00956.

### Statistical Analysis

Patient characteristics of the two cohorts are presented as a number (frequency) and percentage for categorical variables and either mean (standard deviation) or mean (range) for continuous variables. Statistical differences for categorical variables were assessed using a Kruskal-Wallis test (nonparametric) for cohort 1, and Mann-Whitney t-test in cohort 2. For cohort 1, an ANCOVA analysis adjusted for age and gender was used to calculate the differences between the groups of patients. For cohort 2, an ANCOVA analysis adjusted for age was used to calculate the differences between the groups of patients ranging from 4-90 years of age. Graphs are shown as mean±95% Cl. For all statistical analysis performed, a P-value below 0.05 was considered significant. Statistical analysis and graphs were performed using GraphPad Prism version 9 (GraphPad Software, Inc., La Jolla, CA) and MedCalc version 19.3 (MedCalc Software, Ostend, Belgium).

### Results

### Specificity, Accuracy, and Precision of the C6A6 Assay

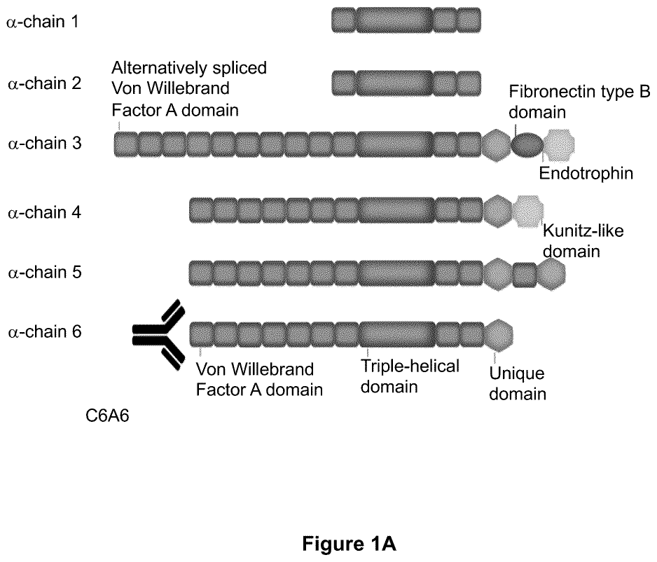

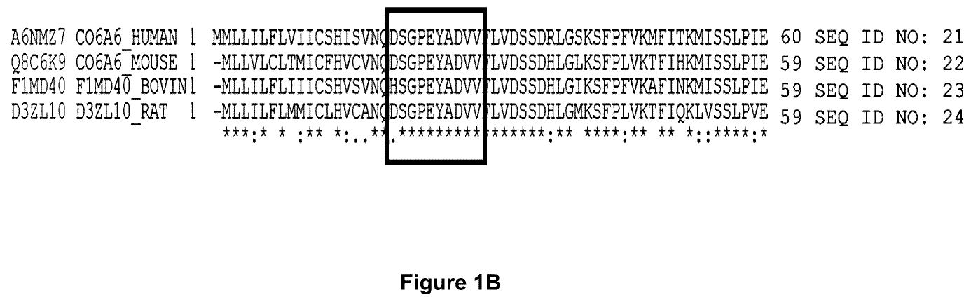

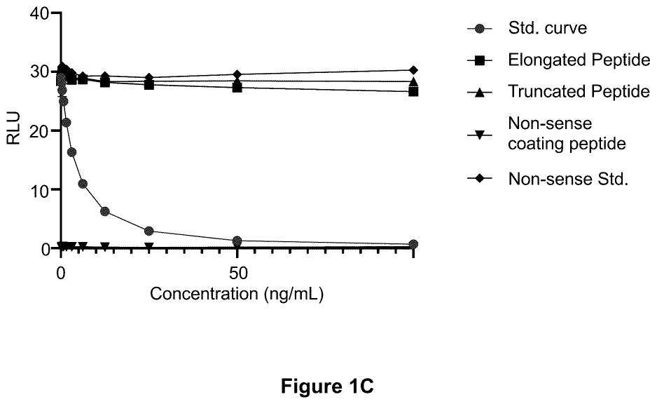

The C6A6 assay uses a monoclonal antibody (mAb) targeting the target sequence DSGPEYADVV (SEQ ID NO: 1) at the N-terminus of the type VI collagen, alpha 6 chain (FIG. 1A). The human target sequence was aligned using UNIPROT with the corresponding mouse, rat and bovine sequences, with the mouse and rat sequences being 100% aligned with the human sequence, while the bovine sequence has one mismatch in position 1 (FIG. 1B). The hybridomas producing the best mAbs were screened for reactivity towards the standard peptide and native material. The clone NBH-306-11 8E7-2C11-1F10-2C10 was chosen for assay development and determined as an IgG1 subtype. To evaluate the specificity of the C6A6 assay, the mAb was tested against the elongated peptide, truncated peptide, non-sense standard peptide and non-sense coater, and showed no reactivity towards those peptides (FIG. 1C). Technical validation was performed to evaluate the novel C6A6 assay, and the technical results are summarized in Table 2. Overall, the C6A6 assay demonstrated a low inter- and intra-variation, good analyte recoveries for 24H at 4° C./20° C. of 93% and 81% respectively, good analyte recoveries for five freeze-thaw cycles of 97%, and with no interference for hemoglobin, lipemia or biotin. Linearity for the human serum samples were observed from 1:3 to 1:16 dilution.

TABLE 2
Summary of technical parameters for the C6A6 assay

| | |
|---|---|
| | RESULTS (Accepted |
| TECHNICAL VALIDATION | recovery: 100 ± 20%) |

| IC50 | 3.86 | ng/mL |
|---|---|---|
| Measurement range (LLMR-ULMR) | 0.60-13.00 | ng/mL |
| Inter-assay variation* | 5% | |
| Intra-assay variation* | 12% | |
| Dilution recovery of human serum* | 105% | |
| Spiking recovery (serum in selection | 104% | |
| peptide)* | | |
| Analyte recovery 24 h, 4° C./20° C.* | 93%/81% | |
| Analyte recovery, 5 freeze/thaw cycles* | 97% | |
| Hemoglobin recovery (low/high)* | 102%/85% | |
| Lipemia recovery (low/high)* | 105%/107% | |
| Biotin interference | <10 | ng/mL |

*Percentages are reported as mean

C6A6 is Elevated in Patients with Dermatological Disorders Compared to Healthy Donors

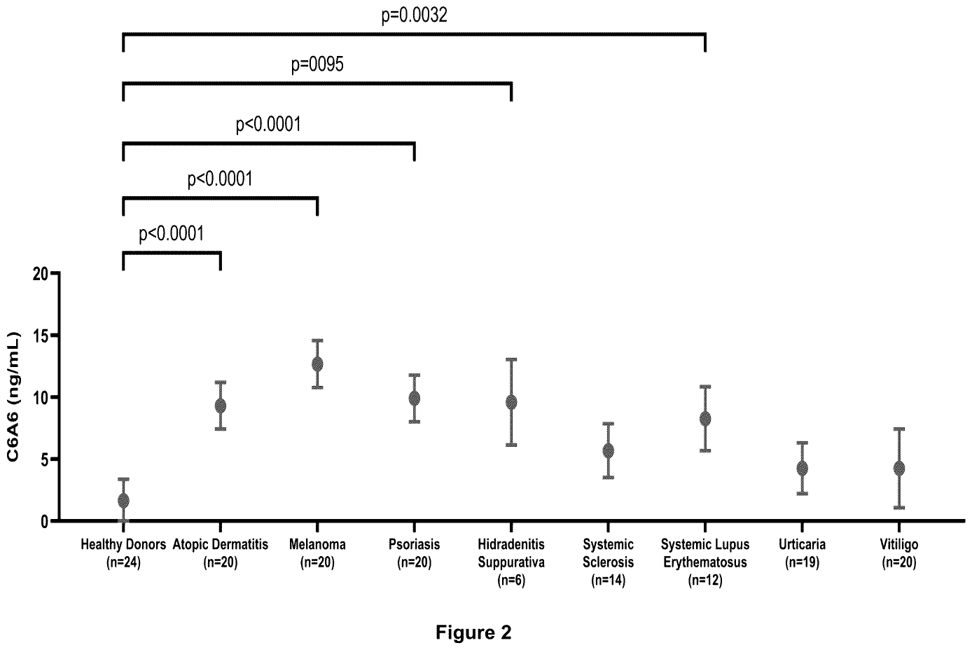

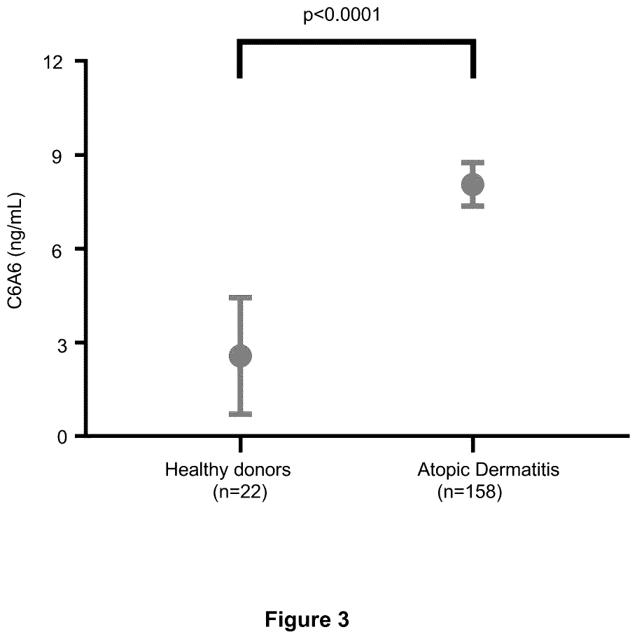

Cohort 1 included patients with atopic dermatitis (mean age: 52.3 years, 10% male), melanoma (mean age: 55.6 years, 10% male), psoriasis (mean age: 50.7 years, 10% male), hidradenitis suppurativa (mean age: 49.5 years, 0% male), systemic sclerosis (mean age: 54.4 years, 5.6% male), systemic lupus erythematosus (mean age: 50.5 years, 16.7% male), urticaria (mean age: 43.6 years, 20% male) and vitiligo (mean age: 56.9 years, 25% male), the patient demographics being shown in Table 3. C6A6 target sequence levels were significantly elevated in serum from patients with atopic dermatitis (p<0.0001), melanoma (p<0.0001), psoriasis (p<0.0001), hidradenitis suppurativa (p=0.0095) and systemic lupus erythemasus (p=0.0032) compared to healthy donors (FIG. 2). No significant difference was found between patients with systemic sclerosis, urticaria, and vitiligo and healthy controls (p=0.304-1.000, respectively).

TABLE 3
Patient demographics for cohort 1. Categorical variables are written as number (percentage),
while continuous variables are mean (standard deviation). BMI, body mass index.

| | | | | | | | Systemic | | | |
|---|---|---|---|---|---|---|---|---|---|---|
| | | Atopic | | | Hidradenitis | Systemic | Lupus | | | |
| | Healthy | dermatitis | Melanoma | Psoriasis | Suppurativa | sclerosis | Erythematosus | Urticaria | Vitiligo | p |
| | (N = 25) | (N = 20) | (N = 20) | (N = 20) | (N = 6) | (N = 18) | (N = 12) | (N = 20) | (N = 8) | value |
| Sex | 11 | 10 | 10 | 10 | 0 | 1 | 2 | 4 | 2 | 0.004 |
| Male | (44.0%) | (50.0%) | (50.0%) | (50.0%) | (0.0%) | (5.6%) | (16.7%) | (20.0%) | (25.0%) | |
| Age | 46.20 | 52.30 | 55.55 | 50.65 | 49.50 | 54.44 | 50.50 | 43.55 | 56.88 | 0.009 |
| Mean (SD) | (11.77) | (4.11) | (9.46) | (6.25) | (13.66) | (11.91) | (5.87) | (16.16) | (12.97) | |
| BMI | NA | NA | 26.69 | 25.71 | NA | 25.57 | 24.74 | NA | NA | — |
| Mean (SD) | | | (6.33) | (1.87) | | (2.68) | (2.10) | | | |
| Ethnicity | | | | | | | | | | — |
| Black, n (%) | 8 | 0 | 0 | 0 | 4 | 0 | 0 | 0 | 0 | |
| | (32.0%) | | | (0.0%) | (66.7%) | (0.0%) | (0.0%) | (0.0%) | (0.0%) | |
| Caucasian, | 17 | 20 | 20 | 20 | 0 | 4 | 12 | 0 | 0 | |
| n (%) | (68.0%) | (100%) | (100%) | (100.0%) | (0.0%) | (22.2%) | (100.0%) | (0.0%) | (0.0%) | |
| Non-Hispanic/ | 0 | 0 | 0 | 0 | 0 | 10 | 0 | 16 | 7 | |
| Latino, n (%) | (0.0%) | | | (0.0%) | (0.0%) | (55.6%) | (0.0%) | (80.0%) | (87.5%) | |
| Unknown, n | 0 | 0 | 0 | 0 | 2 | 4 | 0 | 4 | 1 | |
| (%) | (0.0%) | | | (0.0%) | (33.3%) | (22.2%) | (0.0%) | (20.0%) | (12.5%) | |

C6A6 is Associated with Disease Severity and Suppressed by Immunosuppressant Treatment

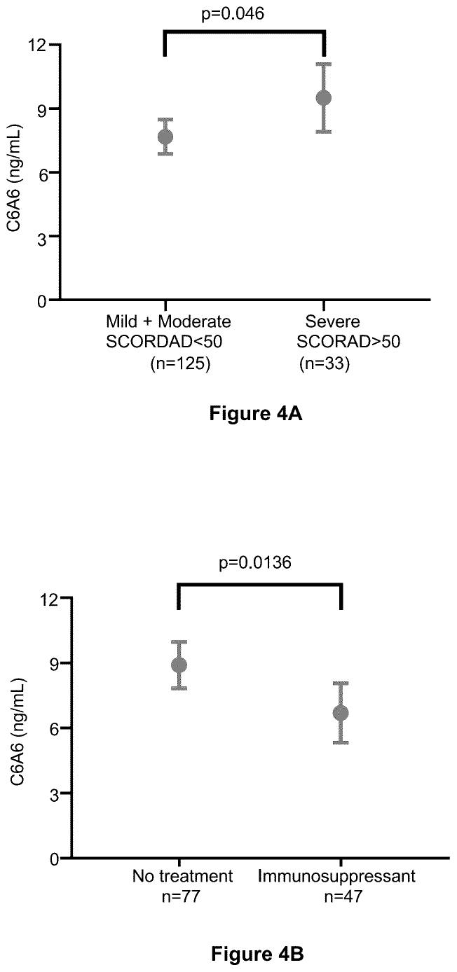

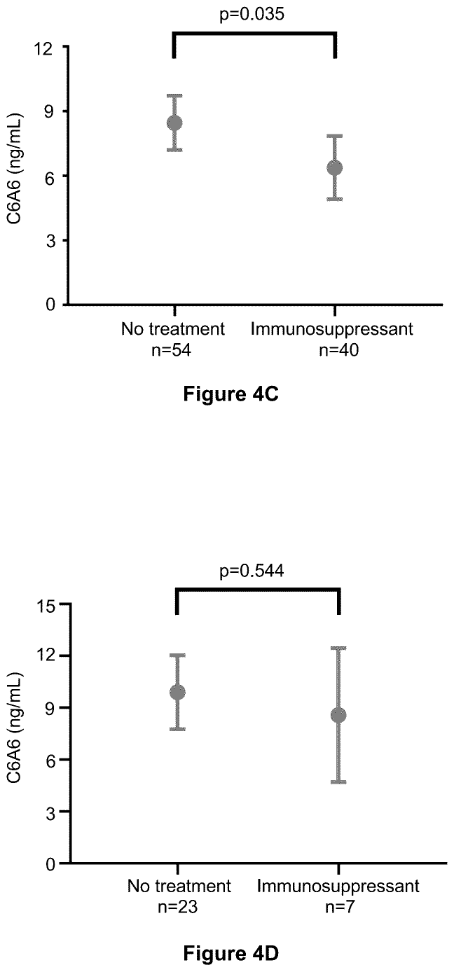

Cohort 2 included 158 patients with atopic dermatitis (mean age: 30.0, 52% male), and 22 healthy donors (mean age: 29.6, 50% male), the patient demographics being shown in Table 4. Patients with atopic dermatitis had significantly higher serum levels of the C6A6 target sequence as compared to healthy controls (p<0.0001). Out of 158 AD patients, 53 (33.3%) patients had mild, 72 (45.3%) had moderate and 33 (21.4%) had severe disease. We evaluated whether C6A6 target sequence levels were associated with disease severity, by comparing patients with severe AD (SCORAD>50) to patients with mild and moderate AD (SCORADO-50). C6A6 levels were significantly higher in patients with severe AD, compared to mild and moderate AD (p=0.046, FIG. 4A). Out of 158 patients, 47 patients received topical immunosuppressant treatment. Patients receiving treatment had significantly lower levels than people not receiving treatment (p=0.0136, FIG. 4B). In addition, patients with mild and moderate AD receiving immunosuppressant treatment had significantly lower levels of C6A6 compared to those without treatment (p=0.035, FIG. 4C), while in patients with severe AD there was no significant difference between those receiving immunosuppressant treatment and those receive no treatment (p=0.544, FIG. 4D).

TABLE 4
Patient Demographics for Cohort 2. Categorical variables
are written as number (percentage), while continuous variables
are mean (standard deviation). BMI, body mass index

| | Healthy donors | | Atopic dermatitis | | |
|---|---|---|---|---|---|
| | (n = 22) | | (n = 158) | | p |
| Males, n (%) | 11 | (50%) | 82 | (52%) | 0.827 |
| Age at sampling, mean | 29.6 | (6.0-65.0) | 30.0 | (4.3-90.9) | 0.902 |
| (range) | | | | | |
| SCORAD, n | | | | | |
| Mild (<25) | | | 53 | (33.5%) | |
| Moderate (25-50) | | | 72 | (45.6%) | |
| Severe (>50) | | | 33 | (20.9%) | |
| Treated with | | | 47 | (29.7%) | |
| Immunosuppressant | | | | | |

### Discussion

The applicant has in this study developed and characterized a competitive ELISA for detection in blood-based samples of a biomarker comprising the N-terminus of COL6α6 using a monoclonal antibody targeting the N-terminus amino acid sequence DSGPEYADVV (SEQ ID NO: 1) (also referred to herein as “C6A6” and/or “the target sequence”). The main results of the study were as follows: 1) the development of a robust and specific assay towards the target sequence DSGPEYADVV (SEQ ID NO: 1); 2) the C6A6 target sequence was detectable in human, mouse and rat serum; 3) elevated levels of C6A6 were present in the sera of patients diagnosed with atopic dermatitis, melanoma, psoriasis, hidradenitis suppurativa and systemic lupus erythematosus, compared to healthy donors; 5) elevated serum levels of C6A6 were associated with severe atopic dermatitis, and the serum levels decreased with immunosuppressant treatment, particularly in patients with mild and moderate atopic dermatitis. To the applicant's knowledge, this is the first study to show that levels of this target sequence can be measured, non-invasively, in serum from patients with dermatological conditions and are associated with disease severity and lowered in patients treated with a topical immunosuppressant in atopic dermatitis.

The C6A6 assay is characterized as a technically robust and accurate assay by showing acceptable dilution recovery, interference, and stability tests. The intra and inter-variation was accepted with values of 5% and 12% respectively. The assay was further characterized as being specific towards the N-terminal target sequence of COL6α6 that is exposed after cleavage of the signal peptide.

In this study, the applicant found that serum levels of C6A6 were suppressed in patients with mild and moderate atopic dermatitis when treated with topical calcineurin inhibitors (TCI). TCIs, including pimecrolimus and tacrolimus, are widely used first-line immunosuppressant topical treatment for atopic dermatitis and psoriasis (3). This shows that serum levels of the target sequence are associated with treatment response. Conversely, no inhibition was seen in patients with severe atopic dermatitis patients, which may indicate this group of patients needs another type of treatment than TCI.

Altogether, this data supports the use of the C6A6 biomarker in the diagnostic and prognostic setting for patients with dermatological conditions, especially atopic dermatitis.

In conclusion, the C6A6 assay showed a high specificity towards the N-terminal target sequence, with levels of this target sequence being elevated in patients with dermatological conditions. The assay was able to distinguish AD patients from healthy donors, demonstrating a high discriminative power. Levels of C6A6 were also upregulated in patients with severe AD, indicating severe patients may experience more fibroblast activity and general tissue remodelling. In addition, it was found that the biomarker levels were lowered in patients treated with topical immunosuppressant treatment (calcineurin inhibitors), indicating this biomarker is associated with treatment response.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used to mean ‘including or consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Simon D, Aeberhard C, Erdemoglu Y, Simon H U. Th17 cells and tissue remodeling in atopic and contact dermatitis. Allergy [Internet] Allergy; 2014 [cited 2022 May 6]; 69:125-31. Available from: pubmed.ncbi.nlm.nih.gov/24372156/2.
2. Dengjel J, Bruckner-Tuderman L, Nyström A. Skin proteomics-analysis of the extracellular matrix in health and disease. Expert Rev Proteomics Taylor & Francis; 2020; 17:377-91.
3. Lynch M D, Watt F M. Fibroblast heterogeneity: implications for human disease. J Clin Invest [Internet] J Clin Invest; 2018 [cited 2022 May 6]; 128:26-35. Available from: pubmed.ncbi.nlm.nih.gov/29293096/4.
4. Cescon M, Gattazzo F, Chen P, Bonaldo P. Collagen VI at a glance. J Cell Sci 2015; 128:3525-31.
5. Wagner M F M G, Theodoro T R, Filho C D A S M, Oyafuso L K M, Pinhal M A S. Extracellular matrix alterations in the skin of patients affected by psoriasis. BMC Mol Cell Biol [Internet] BioMed Central; 2021 [cited 2022 May 6]; 22. Available from:/pmc/articles/PMC8555298/6.
6. Holm Nielsen S, Mortensen J, Willumsen N, Rasmussen D, Mogensen D, Di Sabatino A, et al. A Fragment of Collagen Type VI alpha-3 chain is Elevated in Serum from Patients with Gastrointestinal Disorders. Sci Rep Sci Rep; 2020; 10:5910.
7. Fitzgerald J, Holden P, Hansen U. The expanded collagen VI family: new chains and new questions. Connect Tissue Res [Internet] Connect Tissue Res; 2013 [cited 2022 May 6]; 54:345-50. Available from: pubmed.ncbi.nlm.nih.gov/23869615/8.
8. G T, Z D, AP K, AH B, DA L, KM B, et al. Type VI Collagen Regulates Dermal Matrix Assembly and Fibroblast Motility. J Invest Dermatol [Internet] J Invest Dermatol; 2016 [cited 2021 Sep. 8]; 136:74-83. Available from: pubmed.ncbi.nlm.nih.gov/26763426/9.
9. Dobrota R, Jordan S, Juhl P, Maurer B, Wildi L, Bay-Jensen A C, et al. Circulating collagen neo-epitopes and their role in the prediction of fibrosis in patients with systemic sclerosis: a multicentre cohort study. Lancet Rheumatol [Internet] Elsevier Ltd; 2021; 3: e175-84. Available from: dx.doi.org/10.1016/S2665-9913 (20) 30385-4
10. Jung H J, Heo W II, Park K Y, Lee M K, Ahn J Y, Park M Y, et al. The Role of Collagen VI α6 Chain Gene in Atopic Dermatitis. Ann Dermatol [Internet] Ann Dermatol; 2022 [cited 2022 May 4]; 34:46-54. Available from: pubmed.ncbi.nlm.nih.gov/35221595/11.
11. Ghosh D, Ding L, Sivaprasad U, Geh E, Myers J B, Bernstein J A, et al. Multiple Transcriptome Data Analysis Reveals Biologically Relevant Atopic Dermatitis Signature Genes and Pathways. PLOS One [Internet] PLOS One; 2015 [cited 2022 Mar. 10]; 10. Available from: pubmed.ncbi.nlm.nih.gov/26717000/12.
12. Heo W II, Park K Y, Jin T, Lee M K, Kim M J, Choi E H, et al. Identification of novel candidate variants including COL6A6 polymorphisms in early-onset atopic dermatitis using whole-exome sequencing. BMC Med Genet [Internet] BMC Med Genet; 2017 [cited 2022 Mar. 10]; 18. Available from: pubmed.ncbi.nlm.nih.gov/28125976/13.
13. Gefter M L, Margulies D H, Scharff M D. A simple method for polyethylene glycol-promoted hybridization of mouse myeloma cells. Somatic Cell Genet [Internet] 1977; 3:231-6. Available from: ncbi.nlm.nih.gov/pubmed/605383

1: A method of immunoassay for detecting and/or monitoring a skin disease and/or determining the severity of a skin disease in a patient, the method comprising:
i) contacting a patient sample, selected from blood, serum or plasma, with a monoclonal antibody that specifically binds to the N-terminus amino acid sequence DSGPEYADVV (SEQ ID NO: 1);
ii) detecting and determining the amount of binding between said monoclonal antibody and peptides in the sample; and
iii) correlating said amount of binding with values associated with normal healthy subjects and/or values associated with known disease severity and/or values obtained from said patient at a previous time point and/or with a predetermined cut-off value.

2: The method of claim 1, wherein the monoclonal antibody does not specifically bind to a peptide having the N-terminus amino acid sequence QDSGPEYADVV (SEQ ID NO: 2).

3: The method of claim 1, wherein the monoclonal antibody does not specifically bind to a peptide having the N-terminus amino acid sequence SGPEYADVV (SEQ ID NO: 3).

4: The method of claim 1, wherein the monoclonal antibody is raised against a synthetic peptide having the N-terminus amino acid sequence DSGPEYADVV (SEQ ID NO: 1).

5: The method of claim 1, wherein the skin disease is atopic dermatitis, melanoma, psoriasis, hidradenitis suppurativa or systemic lupus erythematosus.

6: The method of claim 1, wherein the immunoassay is a competition assay or a sandwich assay.

7: The method of claim 1, wherein the immunoassay is a radio-immunoassay or an enzyme-linked immunosorbent assay.

8: An immunoassay kit comprising a monoclonal antibody that specifically binds to the N-terminus amino acid sequence DSGPEYADVV (SEQ ID NO: 1), and at least one of:
a streptavidin coated well plate;
a biotinylated peptide DSGPEYADVV-L-Biotin (SEQ ID NO: 14), wherein L is an optional linker;
a secondary antibody for use in a sandwich immunoassay;
a calibrator protein comprising the N-terminus amino acid sequence DSGPEYADVV (SEQ ID NO: 1);
an antibody biotinylation kit;
an antibody HRP labelling kit;
an antibody radiolabelling kit; or
an assay visualisation kit.

9: The immunoassay kit of claim 8, wherein the monoclonal antibody does not specifically bind to a peptide having the N-terminus amino acid sequence QDSGPEYADVV (SEQ ID NO: 2).

10: The immunoassay kit of claim 8, wherein the monoclonal antibody does not specifically bind to a peptide having the N-terminus amino acid sequence SGPEYADVV (SEQ ID NO: 3).

11: The immunoassay kit of claim 8, wherein the monoclonal antibody is raised against a synthetic peptide having the N-terminus amino acid sequence DSGPEYADVV (SEQ ID NO: 1).

12: A monoclonal antibody that specifically binds to the N-terminus amino acid sequence DSGPEYADVV (SEQ ID NO: 1).

13: The monoclonal antibody of claim 12, wherein the monoclonal antibody does not specifically bind to a peptide having the N-terminus amino acid sequence QDSGPEYADVV SEQ ID NO: 2).

14: The monoclonal antibody of claim 12, wherein the monoclonal antibody does not specifically bind to a peptide having the N-terminus amino acid sequence SGPEYADVV (SEQ ID NO: 3).

15: The monoclonal antibody of claim 12, wherein the monoclonal antibody is raised against a synthetic peptide having the N-terminus amino acid sequence DSGPEYADVV SEQ ID NO: 1).


SEQUENCE LISTING (24 sequences)

SEQ ID NO: 1 - protein, 10 aa, synthetic construct
DSGPEYADVV

SEQ ID NO: 2 - protein, 11 aa, synthetic construct
QDSGPEYADVV

SEQ ID NO: 3 - protein, 9 aa, synthetic construct
SGPEYADVV

SEQ ID NO: 4 - protein, 11 aa, Mus musculus
KASQNVGTDVV

SEQ ID NO: 5 - protein, 7 aa, Mus musculus
SASYRYS

SEQ ID NO: 6 - protein, 9 aa, Mus musculus
QHYDNYPLT

SEQ ID NO: 7 - protein, 5 aa, Mus musculus
SYAMS

SEQ ID NO: 8 - protein, 15 aa, Mus musculus
SVTSGGTHYLDSVKG

SEQ ID NO: 9 - protein, 6 aa, Mus musculus
GVSFAY

SEQ ID NO: 10 - protein, 74 aa, Mus musculus
KASQNVGTDVVWYQQRPGQSPKALMYSASYRYSGVPDRFTGSGSGTDFTLTISNVQSEDLAEYFCQHYDNYPLT

SEQ ID NO: 11 - protein, 72 aa, Mus musculus
SYAMSWVRQTPEKRLEWVASVTSGGTHYLDSVKGRFTISRDIARNILYLQMSSLRSEDTAMYFCARGVSFAY

SEQ ID NO: 12 - protein, 107 aa, Mus musculus
DIVMTQSQKFMSTSVGDRVSVTCKASQNVGTDVVWYQQRPGQSPKALMYSASYRYSGVPDRFTGSGSGTDFTLTISNVQSEDLAEYFCQHYDNYPLTFGAGTKLELK

SEQ ID NO: 13 - protein, 113 aa, Mus musculus
EVKLVESGGDLVKPGGSLKLSCAASGFTFTSYAMSWVRQTPEKRLEWVASVTSGGTHYLDSVKGRFTISRDIARNILYLQMSSLRSEDTAMYFCARGVSFAYWGQGTLVTVSA

SEQ ID NO: 14 - protein, 10 aa, synthetic construct
  MOD_RES 10: note = biotinylated with an optional linker
DSGPEYADVV

SEQ ID NO: 15 - protein, 13 aa, synthetic construct
  MOD_RES 13: note = linked to keyhole limpet hemocyanin
DSGPEYADVVGGC

SEQ ID NO: 16 - protein, 10 aa, synthetic construct
  MOD_RES 10: note = biotinylated
YRDDLKKLLE

SEQ ID NO: 17 - protein, 10 aa, synthetic construct
YRDDLKKLLE

SEQ ID NO: 18 - protein, 11 aa, synthetic construct
  MOD_RES 11: note = biotinylated
DSGPEYADVVK

SEQ ID NO: 19 - protein, 456 aa, Mus musculus
MNFGFSLIFLVLVLNGVQCEVKLVESGGDLVKPGGSLKLSCAASGFTFTSYAMSWVRQTPEKRLEWVASVTSGGTHYLDSVKGRFTISRDIARNILYLQMSSLRSEDTAMYFCARGVSFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 234 aa, Mus musculus
MESQTQVFVYMLLWLSGVDGDIVMTQSQKFMSTSVGDRVSVTCKASQNVGTDVVWYQQRPGQSPKALMYSASYRYSGVPDRFTGSGSGTDFTLTISNVQSEDLAEYFCQHYDNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 60 aa, Homo sapiens
MMLLILFLVIICSHISVNQDSGPEYADVVFLVDSSDRLGSKSFPFVKMFITKMISSLPIE

SEQ ID NO: 22 - protein, 59 aa, Mus musculus
MLLVLCLTMICFHVCVNQDSGPEYADVVFLVDSSDHLGLKSFPLVKTFIHKMISSLPIE

SEQ ID NO: 23 - protein, 59 aa, Bos taurus
MLLILFLIIICSHVSVNQHSGPEYADVVFLVDSSDHLGIKSFPFVKAFINKMISSLPIE

SEQ ID NO: 24 - protein, 59 aa, Rattus norvegicus
MLLILFLMMICLHVCANQDSGPEYADVVFLVDSSDHLGMKSFPLVKTFIQKLVSSLPVE